(19) United States
(12) Patent Application Publication
(10) Pub. No.: US 2023/0181637 A1
(43) Pub. Date: Jun. 15, 2023

(54) NK CELLS AND USES THEREOF FOR TREATMENT OF MICROBIAL INFECTIONS

(71) Applicant: RESEARCH INSTITUTE AT NATIONWIDE CHILDREN'S HOSPITAL, Columbus, OH (US)

(72) Inventors: Dean Anthony LEE, Winchester, OH (US); Brian Patrick TULLIUS, Galloway, OH (US); Meisam Naeimi KARAROUDI, Columbus, OH (US)

(21) Appl. No.: 17/910,992

(22) PCT Filed: Mar. 11, 2021
(86) PCT No.: PCT/US2021/021928

§ 371 Date: Sep. 12, 2022

Related U.S. Application Data
(60) Provisional application No. 62/987,935, filed on Mar. 11, 2020.

(51) Int. Cl. A61K 35/17; C12N 5/0783; A61K 38/17; A61K 38/20; A61K 38/19; A61P 31/14

(52) U.S. Cl. CPC A61K 35/17; C12N 5/0646; A61K 38/1774; A61K 38/2086; A61K 38/20; A61K 38/191; A61P 31/14; C12N 2501/2302; C12N 2501/2315; C12N 2501/2321; C12N 2502/30

(57) ABSTRACT

Disclosed herein are expanded NK cells and methods of using thereof for treating, preventing, reducing, and/or inhibiting a microbial infection.

### CROSS-REFERENCE TO RELATED APPLICATIONS

This application claims the benefit of U.S. Provisional Application No. 62/987,935, filed Mar. 11, 2020, which is incorporated herein by reference in its entirety.

### BACKGROUND

The World Health Organization (WHO) first became aware of the emergence of several cases of pneumonia of an unknown etiology in the city of Wuhan in the Hubei Province of China on Dec. 31, 2019 with Chinese authorities recognizing novel coronavirus (2019-nCoV) as the causative agent on Jan. 7, 2020. Since then, 2019-nCoV has emerged as a growing pandemic with the WHO reporting 117,799,584 confirmed cases and 2,615,018 confirmed deaths worldwide by Mar. 11, 2021. Therefore, what is needed are compositions and methods for treating the 2019-nCoV infection. The compositions and methods disclosed herein address these and other needs.

### SUMMARY

Disclosed herein are methods related to treating, preventing, reducing, and/or inhibiting a microbial infection in a subject comprising administering to the subject a therapeutically effective amount of expanded natural killer (NK) cells.

The details of one or more embodiments of the invention are set forth in the accompanying drawings and the description below. Other features, objects, and advantages of the invention will be apparent from the description and drawings, and from the claims.

In some aspects, disclosed herein are methods of treating, preventing, reducing, and/or inhibiting a microbial infection in a subject, said method comprising administering to the subject a therapeutically effective amount of expanded natural killer (NK) cells, wherein the method further comprising obtaining a nonexpanded, nonactivated NK cell and expanding the nonexpanded, nonactivated NK cell through contacting the nonexpanded, nonactivated NK cell with a plasma membrane vesicle, an exosome, or a feeder cell that is engineered to express membrane bound IL-21. The method disclosed herein generates expanded NK cells with increased expression levels of NK cell receptors (e.g., KIR2DL2, NKp46, NKp44, NKp30, CD226, NKG2D, 2B4, CD11a, OX40, 4-1BB, CD223, and/or ICOS) and anti-microbial effectors (e.g., granzyme B, TNFα, IFNγ, and/or perforin). In some embodiments, the expanded NK cells comprise increased expression levels of KIR2DL2.

In some embodiments, the feeder cell is selected from the group consisting of peripheral blood mononuclear cells (PBMC), RPMI8866, HFWT, 721.221, EBV-LCL, and K562 cell lines.

In some embodiments, the expansion of nonexpanded, nonactivated NK cell occurs ex vivo. In some embodiments, the expansion of the nonexpanded, nonactivated NK cell occurs in vivo. In some embodiments, the nonexpanded, nonactivated NK cell comprises a primary NK cell or an NK cell line. In some embodiments, the expanded NK cells comprise autologous, haploidentical, or allogeneic NK cells.

In some aspects, disclosed herein a method of generating expanded natural killer (NK) cells that comprise increased expression levels of KIR2DL2, comprising obtaining a nonexpanded, nonactivated NK cell and expanding the nonexpanded, nonactivated NK cell through contacting the nonexpanded, nonactivated NK cell with a plasma membrane vesicle, an exosome, or a feeder cell that is engineered to express membrane bound IL-21. In some embodiments, the method further comprises administering a therapeutically effective amount of the expanded NK cells to a subject in need thereof for treating, preventing, inhibitor, and/or reducing a microbial infection.

The methods of any preceding aspect are used for treating, preventing, inhibiting, and/or reducing a microbial infection that comprises a viral infection. In some embodiments, the viral infection comprises a coronavirus infection (such as, for example, avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEM), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV) (SARS-CoV), Severe Acute Respiratory Syndrome (SARS)-Coronavirus (CoV)-2 (SARS-CoV-2, or middle east respiratory syndrome (MERS) coronavirus (CoV) (MERS-CoV), including, but not limited to the B1.351 variant, B.1.1.7 variant, and P.1 variant), a herpes virus infection (such as, for example, Herpes Simplex virus- 1, Herpes Simplex virus-2, Varicella-Zoster virus, Epstein-Barr virus, Cytomegalovirus, Human Herpes virus-6), a polyomavirus, influenza A virus, or influenza B virus. In some embodiments, the coronavirus is 2019-nCoV.

Also disclosed herein is a preclinical method of examining an NK cell adoptive immunotherapy for treating 2019-nCoV infection, comprising administering expanded NK cells to a canine that is infected with 2019-nCoV; and determining that the expanded NK cells are effective if the viral titers of 2019-nCoV in the canine decrease.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows mRNA expression levels in expanded NK cells of proteins known to be involved in the immune response to COVID-19.

FIG. 2 shows fold NK cell expansion. The cells were derived from healthy donors.

FIG. 3 shows change in NK cell phenotype with expansion.

FIG. 4 shows cytokine secretion by primary, IL-15, and IL21-expanded NK cells.

FIG. 5 shows cytokine expression by primary (fresh) and expanded NK (Day 14) cells.

FIG. 6 shows reduced rates of viral activation in the transplant setting on allogeneic transplant protocol including adoptive NK cells (blue) compared to historical controls without receiving NK cells.

FIG. 7 shows increased expression of NKG2D, DNAM-1, NKp30, NKp44, NKp46 in NK cells during expansion.

FIG. 8 shows increased expression of the NKG2C protein (Left) or mRNA (Right) level before and after expansion.

FIG. 9 shows increased IFNγ production upon stimulation.

FIG. 10 shows higher secretion of IFNγ and IL2, and lower secretion of IL8, pentraxin and chitinase in expanded NK cells compared to freshly-isolated NK cells from peripheral blood.

FIG. 11 shows increased chemokine receptors in expanded NK cells compared to freshly-isolated NK cells (including CCR5 and CXCR3) that predict improved trafficking to sites of infection.

FIG. 12 shows phenotype of NK cells expanded on aAPCs bearing membrane-bound cytokines. NK cells purified from PBMC were stimulated weekly with either Clone 4 (mbIL15) or Clone 9.mbIL21 for 3 weeks. Expression of NK cell receptors was determined by flow cytometry. Component subpopulations of fresh NK cells or NK cells expanded on Clone 4 (mbIL15) or Clone 9.mbIL21 from 4 donors as determined by flow cytometry. Mean+/2 SD is shown. P values are for 2-way repeated-measures ANOVA comparing against mbIL21-expanded NK cells with Bonferroni correction (all significant P values are indicated).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Definitions

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

As various changes could be made in the above-described cells and methods without departing from the scope of the invention, it is intended that all matter contained in the above description and in the examples given below, shall be interpreted as illustrative and not in a limiting sense.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., viral titers). It is understood that this is typically in relation to some standard or expected value, in other words it is relative, but that it is not always necessary for the standard or relative value to be referred to. For example, “reduces viral titer” means reducing the viral titer relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Therapeutically effective amount” or “therapeutically effective dose” of a composition (e.g., a composition comprising an agent) refers to an amount that is effective to achieve a desired therapeutic result. In some embodiments, a desired therapeutic result is the control of a microbial infection (e.g., 2019-nCoV infection). In some embodiments, a desired therapeutic result is slowing down disease progression relating to 2019-nCoV infection. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Administration” to a subject includes any route of introducing or delivering to a subject an agent. Administration can be carried out by any suitable route, including oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, palliatively or remedially. Prophylactic treatments are administered to a subject prior to onset (e.g., before the infection occurs), during early onset (e.g., upon initial signs and symptoms relating to the infection), or after an established development of cancer. Prophylactic administration can occur for day(s) to years prior to the manifestation of symptoms of a disease or an infection.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Expanded NK Cells

The current disclosure relates to the use of expanded NK cells to treat, inhibit, reduce, ameliorate, and/or prevent microbial infections.

Natural killer (NK) cells are granular lymphocytes of the innate immune system first described in 1975 for their ability to lyse cancer cells, thus earning them their name as “natural killers”. In addition to their role in tumor immunosurveillance for cancers, natural killer cells have a key role in the immune response to viral infection. NK cells are widely recognized as first responders to viral infections. They recognize target cells through upregulation of stress-related proteins on the surface of virus-infected cells, which trigger direct cytotoxic effector mechanisms as well as cytokine production, both of which are associated with cross-talk to the adaptive immune system. Early NK cell responses are associated with robust adaptive responses, clearance of virus, and clinical recovery. The antiviral activity is mediated by the balance of inhibitory and activating receptors on the NK cells. The inhibitory Killer immunoglobulin-like receptors (KIR) recognize healthy human cells through their expression of major histocompatibility complex (MCH) class I. The interaction between MHC and MR inhibits NK cytolytic activity preventing lysis of healthy human cells. Cells that lack MHC (a common mechanism of viral immune escape) fail to induce an inhibitory signal, tipping the balance toward NK cytolysis of that target. As mentioned above, in addition to the inhibitory receptors such as KIR, NK cells possess activating receptors. These include receptors such as natural-killer group 2, member D (NKG2D) and DNAX accessory molecule-1 (DNAM-1). NKG2D recognizes ligands primarily upregulated by cell stress, including MCH class I chain-related protein A and B (MIC A/B), and UL16 Binding Protein family members 1-6, which bind UL16 protein of human CMV. DNAM-1 recognizes ligands related to poliovirus, specifically the poliovirus receptor (PVR, CD155) and Nectin-2 (CD112, also called PVR2). Virus-specific proteins can also be recognized through an overlapping, but separate group of receptors known as the natural cytotoxicity receptors (NCR). The NCRs include NKp46, NKp44, NKp80, and NKp30. Both Np46 and NKp44 can target capsid structures on virion including influenza, parainfluenza, Sendai disease virus, Newcastle disease virus, and pox viruses. In some embodiments, these expanded NK cells are here termed “hyper-functional NK cells” or “K-NK-cells”.

Natural Killer Cells are a type of cytotoxic lymphocyte of the immune system. NK cells provide rapid responses to virally infected cells and respond to transformed cells. Typically, immune cells detect peptides from pathogens presented by Major Histocompatibility Complex (MHC) molecules on the surface of infected cells, triggering cytokine release, causing lysis or apoptosis. NK cells are unique, however, as they have the ability to recognize stressed cells regardless of whether peptides from pathogens are present on MHC molecules. They were named “natural killers” because of the initial notion that they do not require prior activation in order to kill target. In some embodiments, the NK cell of any preceding aspect comprises a primary NK cell or an NK cell line. In some embodiments, the NK cell is a primary NK cell (e.g., NK cells isolated directly from a human or animal tissue). In some embodiments, the non-expanded and non-activated NK cell is a naive NK cell or an NK cell line (e.g., NK-92, NK-YS, KHYG-1, NKL, NKG, SNK-6, or IMC-1). In some embodiments, the NK cell is a CAR-NK cell. In some embodiments, the naïve NK cell is a human NK cell. In some embodiments, the naive NK cell is not a human NK cell. It should be understood herein that the term “naïve NK cell” refers to NK cells having a phenotype that is more characteristic of a quiescent NK cell, for example, lower expression levels of CD11a, NKG2D, and/or NKp46.

Because it is helpful to be able to administer large numbers of immune cells during immunotherapy, in some embodiments the NK cells are expanded NK cells. Expanded NK cells are those that are grown ex vivo in order to grow a large number of NK cells. In some embodiments, the expanded NK cells are autologous cells that can be easily administered to a subject without provoking an immune response. However, in some embodiments, the expanded immune cells are allogeneic immune cells, in which their inherent alloreactivity can be a benefit. In some embodiments, the expanded NK cells are haploidentical. In further embodiments, the expanded NK cells are genetically engineered to include chimeric antigen receptors to help the immune cells target diseased tissue or engineered to include genetic knockouts such as NKp46-KO NK cells, to improve NK cell antiviral activity. Preparation of expanded NK cells includes activating and expanding the NK cells. A number of cytokines (IL-2, IL-12, IL-15, IL-18, IL-21, type I IFNs, and TGF-β) and/or ligands (OX40L or 4-1BBL) have been shown to be useful for contacting nonexpanded, nonactivated NK cells, leading to activating and expanding NK cells ex vivo. For example, in some embodiments, the NK cells used herein are IL-21 expanded NK cells. In some embodiments, the expanded NK cells used herein are NK cells expanded by contacting nonexpanded, nonactivated NK cells with IL-21 in combination with one or more of IL-2, IL-12, IL-15, IL-18. IL-21, type I IFNs, TGF-β, OX40L, and 4-1BBL.

Expansion refers to the proliferation of NK cells so that the population of NK cells is increased. NK cells can be expanded, for example, from peripheral blood mononuclear cells. However, NK cells can also be expanded from other types of cells, such as hematopoietic stem cells or progenitor cells. The initial blood or stem cells can be isolated from a variety of different sources, such placenta, umbilical cord blood, placental blood, peripheral blood, spleen or liver. Expansion occurs in a cell culture medium. Suitable cell culture mediums are known to those skilled in the art. The expanded cells can be a provided as a cell line, which is a plurality of cells that can be maintained in cell culture. Thus, in one aspect, disclosed herein are immunotherapy methods further comprising expanding the at least one NK cell prior to delivering a therapeutically effective amount of the NK cell. In some aspects, the NK cell has been extracted from a subject using known methods prior to performing the method of determining the potency of the NK cell. Alternatively, the NK cell can be sourced from expansion of a cell culture.

The NK cells disclosed herein are expanded and/or activated. In one aspect disclosed herein are NK cells, wherein the cells are expanded in vivo or ex vivo by contacting nonexpanded, nonactivated NK cells with IL-21, IL-15, and/or 4-BBL. In one aspect, the IL-21, IL-15, and/or 4-BBL are provided on the surface of feeder cells, plasma membrane vesicles, liposomes, and/or exosomes. In one aspect, the IL-21, IL-15, and/or 4-1BBL are provided on the surface of engineered feeder cells, engineered plasma membrane vesicles, engineered liposomes, and/or engineered exosomes. Thus, in one aspect, disclosed herein are expanded NK cells, wherein the NK cells are expanded in vivo or ex vivo by contacting a nonexpanded, nonactivated NK cell with a plasma membrane vesicle, liposome, exosome, or feeder cell that was engineered to express membrane bound IL-21. In some embodiments, the plasma membrane vesicle, liposome, exosome, or feeder cell further comprises 4-BBL, IL-2, IL-15, IL-18, IL-21, type I IFNs, TGF-β. It is shown herein that the expanded NK cells effectively control microbial infections.

Plasma membrane (PM) particles are vesicles made from the plasma membrane of a cell or artificially made (i.e liposomes). A PM particle can contain a lipid bilayer or simply a single layer of lipids. A PM particle can be prepared in single lamellar, multi-lamellar, or inverted form. PM particles can be prepared from Fc-bound feeder cells as described herein, using known plasma membrane preparation protocols or protocols for preparing liposomes such as those described in U.S. Pat. No. 9,623,082, the entire disclosure of which is herein incorporated by reference. In certain aspects, PM particles as disclosed herein range in average diameter from about 100 nm to about 1000 nm, from about 200 to about 500 nm, or from about 170 nm to about 300 nm. In certain aspects, PM particles as disclosed herein range in average diameter of at least about 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, or 1000 nm.

Exosomes are cell-derived vesicles that are present in many and perhaps all eukaryotic fluids. Exosomes contain RNA, proteins, lipids and metabolites that is reflective of the cell type of origin. The reported diameter of exosomes is between 30 and 100 nm. Exosomes are either released from the cell when multivesicular bodies fuse with the plasma membrane or released directly from the plasma membrane. In some embodiments, exosomes are obtained from cancer cells. In some embodiments, the exosomes are leukemic cell exosomes. While this disclosure is given in the context of using exosomes to determine the potency of an immune cell, other extracellular vesicles may also be used to determine the potency of an immune cell. As used herein, the term “extracellular vesicle” includes, but is not limited to, all vesicles released from cells by any mechanism. “Extracellular vesicles” includes exosomes which are released from multivesicular bodies and microvesicles that are shed from the cell surface. “Extracellular vesicles” includes vesicles created by exocytosis or ectocytosis. “Extracellular vesicles” encompasses exosomes released from multivesicular bodies, vesicles released by reverse budding, fission of membrane(s), multivesicular endosomes, ectosomes, microvesicles, microparticles, and vesicles released by apoptotic bodies, and hybrid vesicles containing plasma membrane components. Extracellular vesicles can contain proteins, nucleic acids, lipids, and other molecules common to the originating cell.

In one aspect, the plasma membrane particles, feeder cells, liposomes, or exosomes can be purified from feeder cells that stimulate NK cells. Immune cell stimulating feeder cells for use in the claimed invention, for use in making the engineered plasma membrane particles, engineered feeder cells, engineered liposomes, or engineered exosomes disclosed herein can be either irradiated autologous, haploidentical, or allogeneic peripheral blood mononuclear cells (PBMCs) or nonirradiated autologous or allogeneic PBMCs, RPMI8866, HFWT, 721.221, K562 cells, EBV-LCLs, T cells transfected with one or more membrane bound IL-21, membrane bound IL-15, membrane bound 4-1BBL, membrane bound OX40L, membrane bound TGFβ, and/or membrane bound TNF-α, (such as for example, T cells transfected with membrane bound IL-21, T cells transfected with membrane bound 4-1BBL, T cells transfected with membrane bound IL-15 and 4-1BBL, T cells transfected with membrane bound IL-21 and 4-1BBL), NK cells (including, but not limited to PBMCs, RPMI8866, NK-92, NK-92MI, NK-YTS, NK, NKL, KIL, KIL C.2, NK 3.3, NK-YS, HFWT, K562 cells, autologous cancer cells) transfected with membrane bound IL-21, NK cells (including, but not limited to PBMCs, RPMI8866, NK-92, NK-92MI, NK-YTS, NK, NKL, ML, ML C.2, NK 3.3, NK-YS, HFWT, K562 cells, autologous cancer cells) transfected with membrane bound 4-1BBL, NK cells (including, but not limited to PBMCs, RPMI8866, NK-92, NK-92MI, NK-YTS, NK, NKL, ML, KIL C.2, NK 3.3, NK-YS, HFWT, K562 cells, autologous cancer cells) transfected with membrane bound IL-15 and 4-1BBL , or NK cells (including, but not limited to PBMCs, RPMI8866, NK-92, NK-92MI, NK-YTS, NK, NKL, ML, ML C.2, NK 3.3, NK-YS, HFWT, K562 cells, autologous cancer cells) transfected with membrane bound IL-21 and 4-1BBL as well as other non-HLA or low-HLA expressing cell lines or patient derived primary tumors.

The plasma membrane particle, feeder cells, liposomes, and/or exosomes used in the disclosed methods or to activate and expand the disclosed expanded NK cells, can further comprise additional effector agents to expand and/or activate NK cells. Thus, in one aspect disclosed herein are expanded NK cells, wherein the feeder cells used to generate the disclosed engineered liposomes, engineered exosomes, engineered feeder cells, or engineered plasma membrane particles further comprise at least one additional NK cell effector agent on its cell surface, wherein the at least one additional NK cell effector agent is a cytokine, an adhesion molecule, or an immune cell activating agent (such as, for example, 4-1BBL, IL-2, IL-12, IL-15, IL-18, IL-21, MICA, LFA-1, 2B4, CCR7, OX40L, UBLP2, BCM1/SLAMF2, NKG2D agonists, CD137L, CD137L, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). Accordingly, in one aspect, the feeder cells, liposomes, plasma membrane particles and exosomes generated by said feeder cells can comprise membrane bound versions of any combination of the NK cell activating agents (such as, for example, 4-1BBL, IL-2, IL-12, IL-15, IL-18, IL-21, MICA, LFA-1, 2B4, CCR7, OX40L, UBLP2, BCM1/SLAMF2, NKG2D agonists, CD137L, CD155, CD112, Jagged1, Jagged2, Delta-1, Pref-1, DNER, Jedi, SOM-11, wingless, CCN3, MAGP2, MAGP1, TSP2, YB-i, EGFL7, CCR7, DAP12, and DAP10, Notch ligands, NKp46 agonists, NKp44 agonists, NKp30 agonists, other NCR agonists, CD16 agonists). For example, the exosomes or plasma membrane particles can have IL-15, IL-21, and/or 4-1BBL on their membrane.

In one aspect, the NK cells can be expanded with soluble 4-1BBL, IL-2, IL-12, IL-15, IL-18, IL-21, MICA, LFA-1, 2B4, CCR7, OX40L, UBLP2, BCM1/SLAMF2, NKG2D agonists, CD137L, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists that can be added directly to an ex vivo culture, administered to a subject receiving the NK cells, or secreted by feeder cells, plasma membrane vesicles, liposomes, or exosomes in culture ex vivo or in vivo. Thus, it is understood and herein contemplated that the NK cells can be expanded ex vivo or in vivo.

It is understood and herein contemplated that the NK cells disclosed herein must be exposed to the particle or exosome for a period of time to be induced to produce cytokines. In one aspect, disclosed herein are methods of assaying the potency of an NK cell wherein the NK cell is contacted with an effective amount of a plasma membrane particle, a liposome, or an exosome (including, but not limited to engineered exosomes) for at least 5, 6, 7, 8, 9, 10, 15, 20, 25, 30, 35, 40, 45, 50, 55, 60, 65, 70, 75, 80, 85, 90, 95, 100, 105, 110, 115, 120, 150 minutes, 3, 4, 5,6 7, 8, 9, 10, 11, 12, 13, 14, 15, 16, 17, 18, 19, 20, 21, 22, 23, 24, 30, 32, 36, 42, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 31, 45, 60, 61, 62 days, 3, 4, 5, or 6 months.

In some embodiments, the expanded NK cells disclosed herein comprise increased expression levels of one or more NK cell receptors. In some embodiments, the expanded NK cells comprise increased expression levels of one or more NK cell activating receptors (e.g., KIR2DL2, NKp46, NKp44, NKp30, CD226, NKG2D, 2B4, CD11a, OX40, 4-1BB, CD223, or ICOS). In some embodiments, the expanded NK cells comprise increased expression levels of one or more NK cell inhibitory receptors (e.g., KIR2DL1, KIR3DL1_DS1, NKG2A, BTLA, or TIM-3). In some embodiments, the expanded NK cells comprise increased expression levels of death receptor ligands (GITR, TRAIL, or FASL). In some embodiments, the expanded NK cells comprise increased levels of KIR2DL2. It should be understood and herein contemplated that NKp46 can recognize haemagglutinins on virus-infected cells, triggering NK cell to lyse the infected cells.

In some embodiments, the expanded NK cells disclosed herein comprise increased expression levels of one or more anti-microbial effectors selected from the group consisting of granzyme B, TNFα, IFNγ, and perforin.

### Methods of Generating Expanded NK Cells

In one aspect, disclosed herein are methods of generating expanded natural killer (NK) cells that comprise increased expression levels of KIR2DL2 and methods of increasing the expression of KIR2DL2 in an NK cell, comprising obtaining a nonexpanded, nonactivated NK cell and expanding the nonexpanded, nonactivated NK cell through contacting the nonexpanded, nonactivated NK cell with IL-21, IL-15, and/or 4-BBL. In one aspect, methods of generating expanded natural killer (NK) cells that comprise increased expression levels of KIR2DL2, wherein the cells are expanded in vivo or ex vivo by contacting nonexpanded, nonactivated NK cells with IL-21, IL-15, and/or 4-1BBL. In one aspect, the IL-21, IL-15, and/or 4-1BBL are provided on the surface of feeder cells, plasma membrane vesicles, liposomes, and/or exosomes. In one aspect, the IL-21, IL-15, and/or 4-1BBL are provided on the surface of engineered feeder cells, engineered plasma membrane vesicles, engineered liposomes, and/or engineered exosomes. Thus, in one aspect, disclosed herein are expanded NK cells, wherein the NK cells are expanded in vivo or ex vivo by contacting a nonexpanded, nonactivated NK cell with a plasma membrane vesicle, liposome, exosome, or feeder cell that was engineered to express membrane bound IL-21. In some embodiments, the plasma membrane vesicle, liposome, exosome, or feeder cell further comprises 4-1BBL, IL-2, IL-15, IL-18, IL-21, type I IFNs, TGF-β. It is shown herein that the expanded NK cells effectively control microbial infections.

In one aspect, the plasma membrane particles, feeder cells, liposomes, or exosomes used in the disclosed methods of generating expanded NK cells comprising increased expression levels of KIR2DL2 and methods of increasing expression of KIRDL2 can be purified from feeder cells that stimulate NK cells. Immune cell stimulating feeder cells for use in the claimed invention, for use in making the engineered plasma membrane particles, engineered feeder cells, engineered liposomes, or engineered exosomes disclosed herein can be either irradiated autologous, haploidentical, or allogeneic peripheral blood mononuclear cells (PBMCs) or nonirradiated autologous or allogeneic PBMCs, RPMI8866, HFWT, 721.221, K562 cells, EBV-LCLs, T cells transfected with one or more membrane bound IL-21, membrane bound IL-15, membrane bound 4-1BBL, membrane bound OX40L and/or membrane TNF-α, (such as for example, T cells transfected with membrane bound IL-21, T cells transfected with membrane bound 4-1BBL, T cells transfected with membrane bound IL-15 and 4-1BBL , T cells transfected with membrane bound IL-21 and 4-1BBL), NK cells (including, but not limited to PBMCs, RPMI8866, NK-92, NK-92MI, NK-YTS, NK, NKL, ML, KIL C.2, NK 3.3, NK-YS, HFWT, K562 cells, autologous cancer cells) transfected with membrane bound IL-21, NK cells (including, but not limited to PBMCs, RPMI8866, NK-92, NK-92MI, NK-YTS, NK, NKL, ML, KIL C.2, NK 3.3, NK-YS, HFWT, K562 cells, autologous cancer cells) transfected with membrane bound 4-1BBL, NK cells (including, but not limited to PBMCs, RPMI8866, NK-92, NK-92MI, NK-YTS, NK, NKL, KIL, KIL C.2, NK 3.3, NK-YS, HFWT, K562 cells, autologous cancer cells) transfected with membrane bound IL-15 and 4-1BBL , or NK cells (including, but not limited to PBMCs, RPMI8866, NK-92, NK-92MI, NK-YTS, NK, NKL, ML, KIL C.2, NK 3.3, NK-YS, HFWT, K562 cells, autologous cancer cells) transfected with membrane bound IL-21 and 4-1BBL as well as other non-HLA or low-HLA expressing cell lines or patient derived primary tumors.

As used in the nonexpanded, nonactivated NK cell used in the disclosed methods of generating expanded NK cells comprising increased expression levels of KIR2DL2 and methods of increasing expression of KIRDL2 in an NK cell comprises a primary NK cell, CAR-NK cell, memory-like NK cell, or an NK cell line.

In one aspect the expanded NK cells generated by the disclosed methods can comprise increased expression levels of one or more NK cell receptors selected from group consisting of KIR2DL2, NKp46, NKp44, NKp30, CD226, NKG2D, 2B4, CD11a, OX40, 4-1BB, CD223, and ICOS. Additionally, the expanded NK cells comprise increased expression levels of one or more anti-microbial effectors selected from group consisting of granzyme B, TNFα, IFNγ, and perforin.

### Methods of Treating Microbial Infection

Characteristics of patients initially infected with 2019-nCoV in Wuhan are shown in Table 1. The most common manifestation of the virus is fever with over a third of patients having fever >39° C. Cough and fatigue were also quite common. More than half of patients developed dyspnea and nearly one third had respiratory distress severe enough to require ICU management. The disease progresses rapidly with the median time from onset of symptoms to hospitalization being 7 days, to dyspnea being 8 days, to ARDS 9 days, and to need for mechanical ventilation 10.5 days.

TABLE 1
clinical manifestations of 2019-nCoV
Table 1: Demographics and baseline characteristics of patients infected with 2019-nCoV

| | All patients | | ICU care | | No ICU care | | |
|---|---|---|---|---|---|---|---|
| | (n = 41) | | (n = 13) | | (n = 28) | | p value |
| Characteristics | | | | | | | |
| Age, years | 49.0 | (41.0-58.0) | 49.0 | (41.0-61.0) | 49.0 | (41.0-57.5) | 0.60 |
| Sex | — | | — | | — | | 0.24 |
| Men | 30 | (73%) | 11 | (85%) | 19 | (68%) | — |
| Women | 11 | (27%) | 2 | (15%) | 9 | (32%) | — |
| Huanan seafood market | 27 | (66%) | 9 | (69%) | 18 | (64%) | 0.75 |
| exposure | | | | | | | |
| Current smoking | 3 | (7%) | 0 | | 3 | (11%) | 0.31 |
| Any comorbidity | 13 | (32%) | 5 | (38%) | 8 | (29%) | 0.53 |
| Diabetes | 8 | (20%) | 1 | (8%) | 7 | (25%) | 0.16 |
| Hypertension | 6 | (15%) | 2 | (15%) | 4 | (14%) | 0.93 |
| Cardiovascular disease | 6 | (15%) | 3 | (23%) | 3 | (11%) | 0.32 |
| Chronic obstructive | 1 | (2%) | 1 | (8%) | 0 | | 0.14 |
| pulmonary disease | | | | | | | |
| Malignancy | 1 | (2%) | 0 | | 1 | (4%) | 0.49 |
| Chronic liver disease | 1 | (2%) | 0 | | 1 | (4%) | 0.68 |
| Signs and symptoms | | | | | | | |
| Fever | 40 | (98%) | 13 | (100%) | 27 | (96%) | 0.68 |
| Highest temperature, ° C. | — | | — | | — | | 0.037 |
| <37.3 | 1 | (2%) | 0 | | 1 | (4%) | — |
| 37.3-38.0 | 8 | (20%) | 3 | (23%) | 5 | (18%) | — |
| 38.3-39.0 | 18 | (44%) | 7 | (54%) | 11 | (39%) | — |
| >39.0 | 14 | (34%) | 3 | (23%) | 11 | (39%) | — |
| Cough | 31 | (76%) | 11 | (85%) | 20 | (71%) | 0.35 |
| Myalgia or fatigue | 18 | (44%) | 7 | (54%) | 11 | (39%) | 0.38 |
| Sputum production | 11/39 | (28%) | 5 | (38%) | 6/26 | (23%) | 0.32 |
| Headache | 3/38 | (8%) | 0 | | 3/25 | (12%) | 0.10 |
| Haemoptysis | 2/39 | (5%) | 1 | (8%) | 1/26 | (4%) | 0.46 |
| Diarrhoea | 1/38 | (3%) | 0 | | 1/25 | (4%) | 0.66 |
| Dyspnoea | 22/40 | (55%) | 12 | (92%) | 10/27 | (37%) | 0.0010 |
| Days from illness onset to | 8.0 | (5.0-13.0) | 8.0 | (6.0-17.0) | 6.5 | (2.0-10.0) | 0.22 |
| dyspnoea | | | | | | | |
| Days from first admission | 5.0 | (1.0-8.0) | 8.0 | (5.0-14.0) | 1.0 | (1.0-6.5) | 0.0023 |
| to transfer | | | | | | | |
| Systolic pressure, mm Hg | 125.0 | (119.0-135.0) | 145.0 | (123.0-167.0) | 122.0 | (118.5-129.5) | 0.018 |
| Respiratory rate >24 | 12 | (29%) | 8 | (62%) | 4 | (14%) | 0.0023 |
| breaths per min | | | | | | | |

Data are median (IQR).
n (%), or n/N (%), where N is the total number of patients with available data.
p values comparing ICU care and no ICU care are from χ² test, Fisher's exact test, or Mann-Whitney U test.
2019-nCoV-2019 novel coronavirus.
ICU = intensive care unit.

The recombinant spike protein from the Middle East Respiratory Syndrome Coronavirus (MERS-CoV) in a mouse model leads within 24 hours to up-regulation of cytokines and chemokines known to be related to activation of NK cells for cytolysis of virally infected cells. The spike protein from 2019-nCoV is similarly antigenic. IgG against the SARSCoV spike protein can tightly bind the spike protein from 2019-nCoV. This ability of the adaptive immune system to produce a humoral immune response effective against 2019-nCoV augments the innate NK response through antibody-dependent cellular cytotoxicity (ADCC).

SARS is caused by a similar pandemic coronavirus (SARS-CoV). There is a defect in innate immunity that occurs in SARS-CoV infected patients with a demonstrably lower number of circulating NK cells in blood from SARS-CoV patients than in blood from patients infected with M pneumoniae pneumonia. Additionally, survival from severe SARS correlated significantly not only with the density of NK cells in the peripheral blood, but specifically with the percentage of these NK cells that are CD158b+ (KIR2DL2). Given the similarity both clinically and structurally between SARS-CoV and 2019-nCoV, augmentation of innate NK cell immunity through an adoptive NK cellular therapy confers a survival benefit to patients infected with 2019-nCoV. Importantly, increased CD158b expression on NK cells is more prevalent in children compared to healthy adults. SARS-CoV and 2019-nCoV infections in children are generally much less severe than that of adults. This provides evidence for a function of anti-coronaviral surveillance from this subset of NK cells. KIR2DL2 is highly linked to KIR-B genotypes, which contain more activating MR receptors and are associated with increased NK cell function. However, it is not clear whether this protection is mediated directly by NK cells expressing KIR2DL2, or whether KIR2DL2 is a surrogate marker of increased activating receptors, or of increased licensing of NK cells, or of Group C1 HLA alleles mediating protection through adaptive immunity.

It is understood and herein contemplated that the expanded NK cells disclosed herein comprises increased expression levels of NK cell activating receptors, such as KIR2DL2 that is a NK cell receptor relating to enhanced NK cell anti-microbial cytotoxic effects. Accordingly, in some aspects, disclosed herein is a method of treating, preventing, reducing, and/or inhibiting a microbial infection in a subject comprising administering to the subject a therapeutically effective amount of expanded natural killer (NK) cells.

In some aspects, disclosed herein is method of generating expanded natural killer (NK) cells that comprise increased expression levels of KIR2DL2, comprising obtaining a nonexpanded, nonactivated NK cell and expanding the nonexpanded, nonactivated NK cell through contacting the nonexpanded, nonactivated NK cell with a plasma membrane vesicle, an exosome, or a feeder cell that is engineered to express membrane bound IL-21.

As the timing of a microbial infection (e.g., 2019-nCoV infection) can often not be predicted, it should be understood the disclosed methods of treating, preventing, reducing, and/or inhibiting a microbial infection (e.g., 2019-nCoV infection) can be employed prior to the infection, or following the infection but prior to or following onset of the symptoms related to the infection. In one aspect, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years;12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the microbial infection; ; concurrently with the infection; 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months after the infection, but prior to onset of any symptoms of the infection; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the microbial infection or after onset of any symptoms of the infection.

Dosing frequency for the compositions disclosed herein (e.g., expanded NK cells), includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiment, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiment, the dosing frequency for the compositions (e.g., expanded NK cells) includes, but is not limited to, at least once a day, twice a day, or three times a day. In some embodiment, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiment, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiment, the interval between each administration is constant. For example, the administration can be carried out daily, every two days, every three days, every four days, every five days, or weekly. Administration can also be continuous and adjusted to maintaining a level of the compound within any desired and specified range. Each dose can comprise at least about 1×10⁸ NK cells/kg (e.g, at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ NK cells/kg).

In some embodiments, the method of treating, inhibiting, reducing, ameliorating and/or preventing a microbial infection disclosed herein further comprises obtaining a NK cell and expanding the nonexpanded, nonactivated NK cell through contacting the nonexpanded, nonactivated NK cells with a plasma membrane vesicle, an exosome, or a feeder cell that is engineered to express membrane bound IL-21. The expansion of the nonexpanded, nonactivated NK cell can occur ex vivo and/or in vivo. In some embodiments, the NK cell of any preceding aspect comprises a primary NK cell or an NK cell line. In some embodiments, the NK cell is a primary NK cell (e.g., NK cells isolated directly from a human or animal tissue). In some embodiments, the NK cell is an NK cell line (e.g., NK-92, NK-YS, KHYG-1, NKL, NKG, SNK-6, or IMC-1). In some embodiments, the NK cell is a CAR-NK cell. The primary NK cell or CAR-NK cell can be derived from the subject or not from the subject. In some embodiments, the nonexpanded, nonactivated NK cell is a human NK cell. In some embodiments, the nonexpanded, nonactivated NK cell is not a human NK cell.

In some embodiments, the expanded NK cells comprise increased expression levels of one or more NK cell receptors selected from group consisting of KIR2DL2, NKp46, NKp44, NKp30, CD226, NKG2D, 2B4, CD11a, OX40, 4-1BB, CD223, and ICOS. In some embodiments, the expanded NK cells comprise increased expression levels of KIR2DL2. In some embodiments, the expanded NK cells comprise increased expression levels of NKp46.

In some embodiments, the expanded NK cells comprise increased expression levels of one or more anti-microbial effectors selected from group consisting of granzyme B, TNFα, IFNγ, and perforin.

In one aspect disclosed herein are methods of treating, inhibiting, reducing, ameliorating, and/or preventing a viral infection in a subject comprising administering to any of the expanded NK cells disclosed herein.

For example, disclosed herein are methods of treating, inhibiting, reducing, ameliorating, and/or preventing a microbial infection, wherein the microbial infection is a viral infection, wherein the viral infection comprises an infection of Herpes Simplex virus-1, Herpes Simplex virus-2, Varicella-Zoster virus, Epstein-Barr virus, cytomegalovirus, Human Herpes virus-6, polyomavirus, or avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2), middle east respiratory syndrome (MERS) coronavirus (CoV) (MERS-CoV)), influenza A virus, or influenza B virus.

As noted above, the viral infection can be a coronavirus infection. Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 27 to 34 kilobases. The structure of coronavirus generally consists of the following: spike protein, hemagglutinin-esterease dimer (HE), a membrane glycoprotein (M), an envelope protein (E) a nucleoclapid protein (N) and RNA. The coronavirus family comprises genera including, for example, alphacoronavius (e.g., Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512), betacoronavirus (e.g., 2019-nCoV, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus (MERS). Human coronavirus 0C43, Hedgehog coronavirus 1 (EriCoV)), gammacoronavirus (e.g., Beluga whale coronavirus SW1, Infectious bronchitis virus), and deltacoronavirus (e.g., Bulbul coronavirus HKU11, Porcine coronavirus HKU15). In some embodiments, the virus infection is 2019-nCoV infection (including, but not limited to the B1.351 variant, B.1.1.7 variant, and P.1 variant). In some embodiments, the virus infection is severe acute respiratory syndrome-related coronavirus (SARS) infection. In some embodiments, the virus infection is MERS coronavirus infection.

It should be understood and herein contemplated that “2019-nCoV”, “severe acute respiratory syndrome coronavirus 2”, or “SARS-CoV-2” are used interchangeably herein to refer to the coronavirus that the cause coronavirus disease COVID-19, according to the definition given by World Health Organization. The COVID-19 comprises symptoms including, for example, fever, cough, short of breath, lymphocytopenia, lung inflammation and/or ground-glass opacity on chest computed tomography.

Accordingly, in some embodiments, disclosed herein is a method of treating, preventing, inhibiting and/or reducing 2019-nCoV in a subject comprising administering to the subject a therapeutically effective amount of expanded natural killer (NK) cells.

In some embodiments, disclosed herein is a method of treating, preventing, inhibiting and/or reducing COVID-19 in a subject comprising administering to the subject a therapeutically effective amount of expanded natural killer (NK) cells.

Where the treatment methods is designed to treat a viral infection, it is understood an herein contemplated that the therapeutic agent comprises an antiviral agent selected from the group comprising remdesivir, acyclovir, famciclovir, valacyclovir, penciclovir, ganciclovir, ritonavir, lopinavir, saquinavir, and the like; cimetidine; ranitidine; captopril; metformin; bupropion; fexofenadine; oxcarbazepine; leveteracetam; tramadol; and/or any of their isomers tautomers, analogs, polymorphs, solvates, derivatives, or pharmaceutically acceptable salts.

Polyomaviruses are unenveloped double-stranded DNA viruses that generally cause asymptomatic infection in healthy individuals. However, polyomaviruses can cause serious disease in immunocompromised individuals. In some embodiments, the virus infection is BV virus infection. In some embodiments, the virus infection is Merkel cell polyomavirus (MCV) virus infection. In some embodiments, the virus is JC virus infection. In some embodiments, the virus infection is simian vacuolating virus 40 (SV40) infection.

### Preclinical Methods

In some aspects, disclosed herein is a preclinical method of examining an NK cell adoptive immunotherapy for treating 2019-nCoV infection, comprising a) administering expanded canine NK cells to a canine that is previously infected with 2019-nCoV; and b) determining that the expanded NK cells are effective if the viral titers of 2019-nCoV in the canine decrease.

In some embodiments, the preclinical method further comprising obtaining a nonexpanded, nonactivated NK cell and expanding the nonexpanded, nonactivated NK cell through contacting the nonexpanded, nonactivated NK cell with a plasma membrane vesicle, an exosome, or a feeder cell that is engineered to express membrane bound IL-21.

In some embodiments, the nonexpanded, nonactivated NK cell comprises a primary NK cell or an NK cell line. In some embodiments, the nonexpanded, nonactivated NK cell is a canine NK cell.

### Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid—or base—addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### NK Cell Resistance to 2019-nCoV Infection

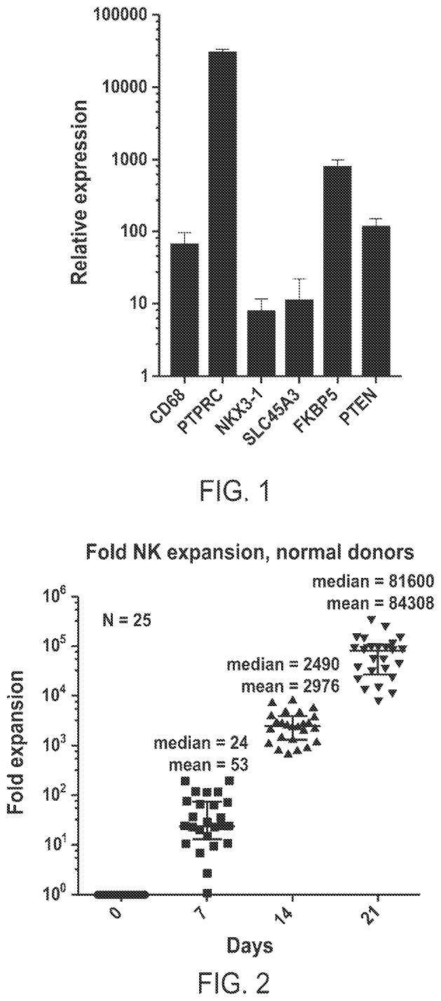

2019-nCoV infection of human tissues is mediated by the interaction of glycoprotein-S on the viral capsid and ACE-2, highly expressed on cells of the respiratory tract. RNA analysis of expanded NK cells reveals a total lack of ACE-2 expression (no detectable transcript for ACE2, AGER, SFTPC, SCGB3A2, TPPP3, AR, TMPRSS4, ETV1, ERG, ETV4, FAM3B). However, expanded NK cells are known to have high relative expression of proteins involved as epitopes for immune engagement of COVID-19 including CD68, PTPRC, NKX3-1, SLC45A3, and PTEN (FIG. 1).

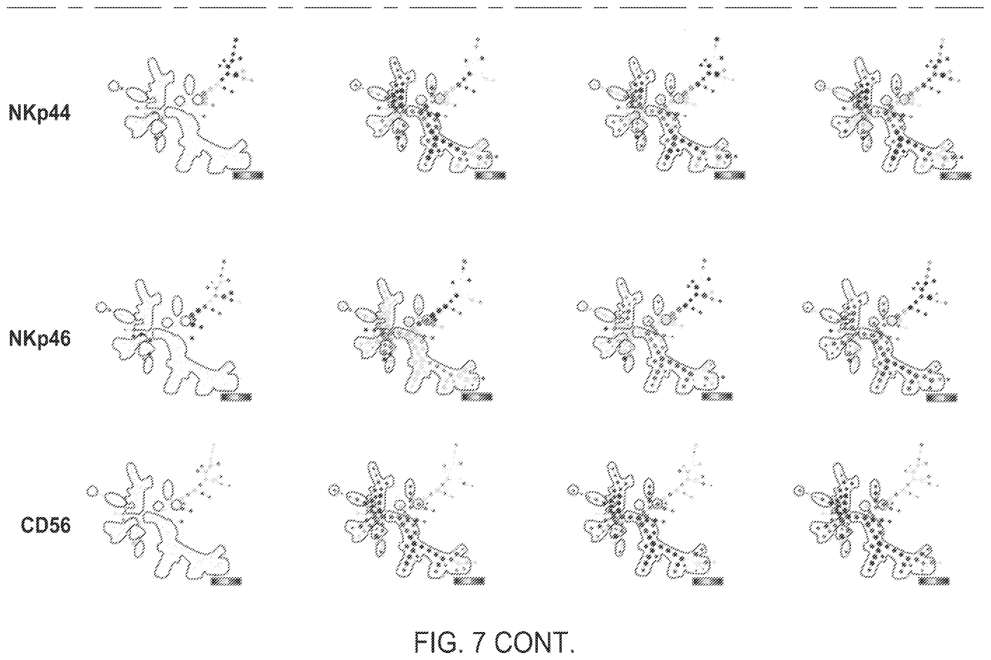

NK cells target and kill virus-infected cells through recognition of different stress-related and viral proteins on the surface, rather than a single protein or antigen. Viral proteins can be recognized by receptors known as natural cytotoxicity receptors (NCR), which include NKp46, NKp44, and NKp30 (FIG. 7), and which are known to target influenza, parainfluenza, Sendai disease virus, Newcastle disease virus, and pox viruses. NK cells naturally adapt to new viral infections, with durable changes to the profile of NK-cell phenotypes in recovered patients.

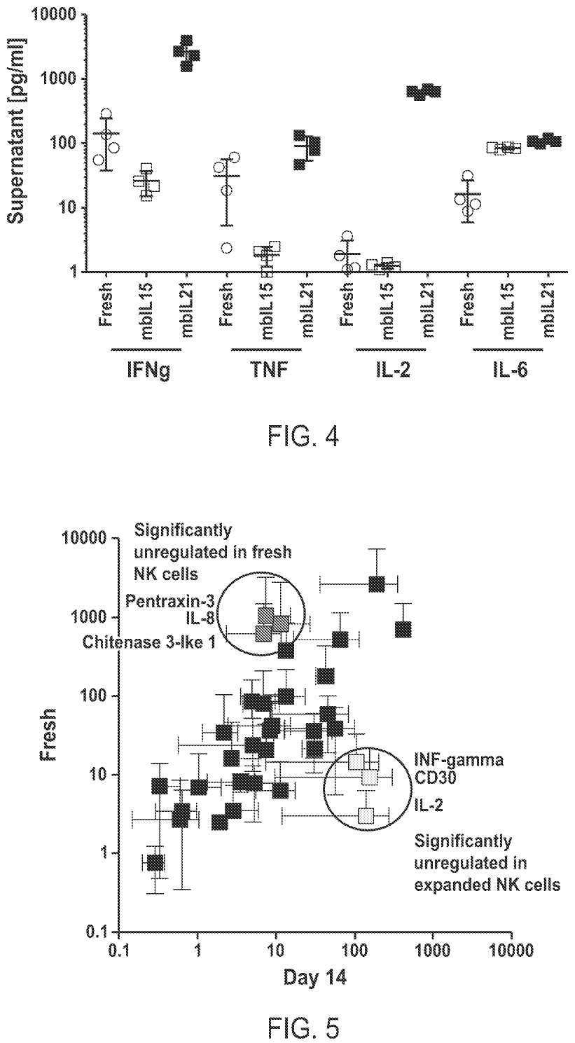

The interferons (IFNs) were discovered as antiviral molecules that interfere with viral infection. Unlike the other IFNs, IFN-γ is produced only by T and NK cells. IFN-γ suppresses viral entry, replication, gene expression, stability, release, and reactivation for most virus classes including that of coronavirus (FIGS. 4 and 5).

### Example 2

### NK Cell Expansion

NK cells have now been successfully and safely utilized as an adoptive immunotherapy for the past 30 years. A platform for expansion of primary NK cells has been developed utilizing K562 feeder cells expressing membrane-bound IL-21 (mbIL21) and ex vivo growth and activation of NK cells with soluble, recombinant IL-2. Activation of STAT3 in NK cells by IL-21 stimulation allows for continued log-phase, fold expansion of activated NK cells to 80,000 in 3 weeks (FIG. 2). This retained expansion potential—not typically a feature of mature NK cells—is related to preservation of telomere length, preventing cell senescence.

### Example 3

### Enhanced Anti-Viral Activity

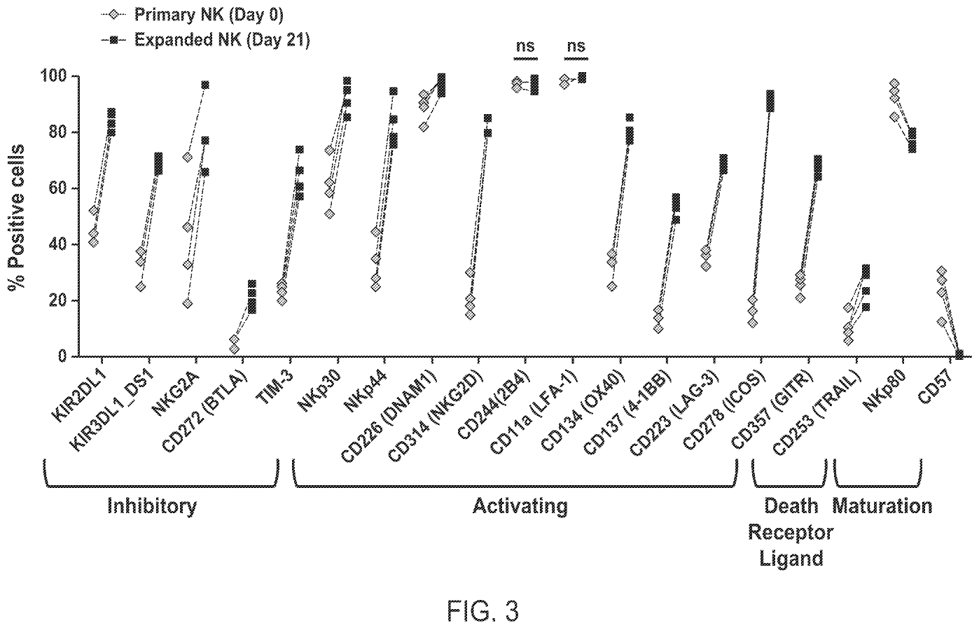

In addition to the necessary fold expansion required to reach a cell quantity sufficient for a viable adoptive cell therapy, this expansion platform activates the NK cells in a manner that enhances them for anti-cancer and also anti-viral cytolytic activity compared to primary NK cells. The NCRs NKp46, NKp44, and NKp30 are all significantly upregulated on expanded NK cells compared to primary NK cells (FIGS. 3 and 7). Specifically, expanded NK cells display the upregulated KIR2DL2/3 expression that is associated with a favorable phenotype for SARS-CoV and SARS-CoV-2 survival (FIG. 12). These favorable phenotypic changes pair with functional enhancement as well. Perforin and granzyme B are both upregulated in expanded NK cells, granting them enhanced cytolytic activity against cellular and viral targets.

Cytokine secretion is also important for antiviral efficacy. Interferon gamma (IFNγ) has been shown to inhibit replication of RNA viruses including SARS-CoV and SARS-CoV-2. This antiviral cytokine is significantly upregulated in mbIL21-expanded NK cells compared both to primary NK cells (see FIG. 4 and FIG. 5) and NK cells expanded with an IL-15 expressing feeder cell. This enhanced cytokine secretion can provide an additional mechanism beyond direct lysis and ADCC for suppressing COVID-19 infection, as compared to mild cases, severe COVID-19 patients contained more macrophages, but less proportion of T and NK cells.

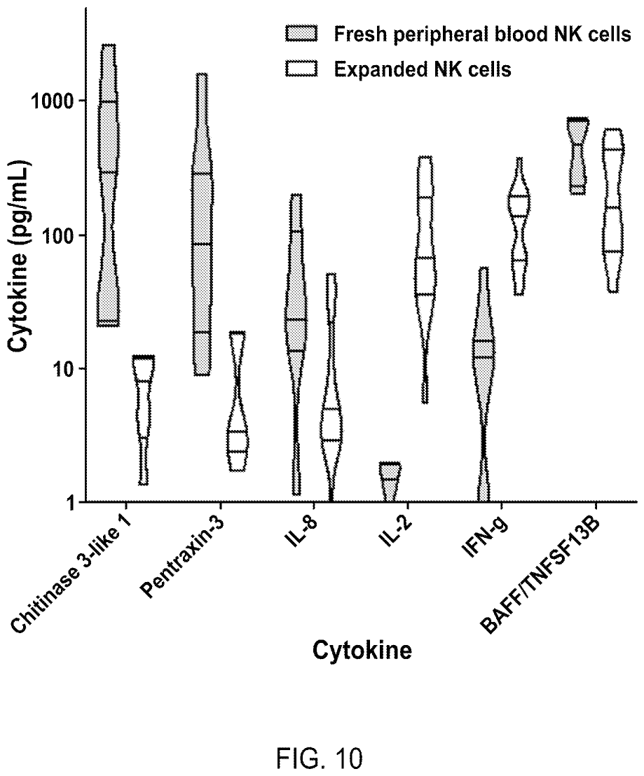

Hyperfunctional K-NK cells were generated using mbIL21 feeder cell membrane particles to proliferate and activate these cells. K-NK cells have high anti-tumor and anti-viral cytolytic activity, consist of a high percentage of NKG2C+ and CD158b+ NK cells, and have an upregulated metabolism showing no signs of exhaustion after 9 weeks in culture. In fact, the metabolism of K-NK cells allows the NK cells to thrive in low nutrient and low oxygen environments. K-NK cells hypersecrete IFNγ to suppress viral replication, and have reduced secretion of cytokines such as IL-6, IL-8, pentraxin (associated with toxic shock and sepsis), and IL-8 (associated with neutrophil recruitment), and chitinase (associated with pathogenic tissue inflammation, fibrosis, and asthma) that are associated with tissue damage or worse outcomes in COVID-19 (FIG. 10). Additionally, K-NK cells have similar levels of B-cell activating factor (BAFF), which is important for B cells responses

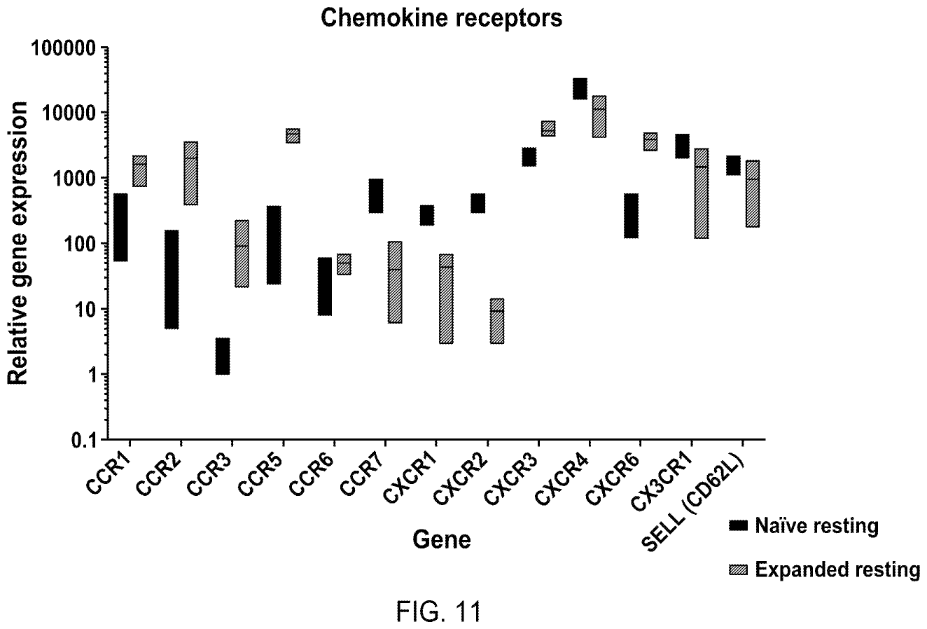

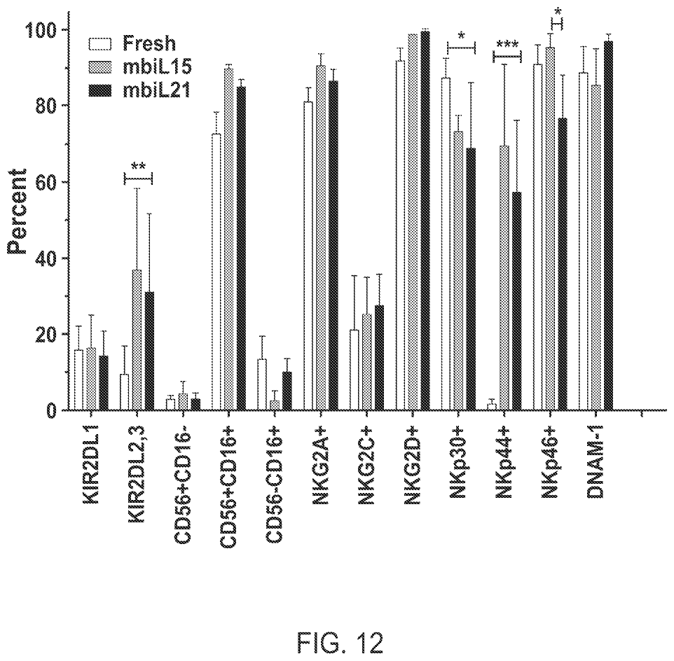

Influenza infection causes potentially lethal pneumonia. Shortly after influenza infection, NK cells become hyperresponsive with an increased killing of influenza-infected cells, facilitated by the activating NKp44 and NKp46 receptor on NK cells, which recognize viral HA on the surface of infected cells. NK cells are actively recruited to the lungs and airways during influenza infection. Infected respiratory epithelial cells release chemokines that attract NK cells. Migration of NK cells is dictated by the severity of influenza infection, and partially dependent on CXCR3 and CCR5 receptors on NK cells and their ligands (FIG. 11).

### Example 4

### NK Cells as Adoptive Immunotherapy

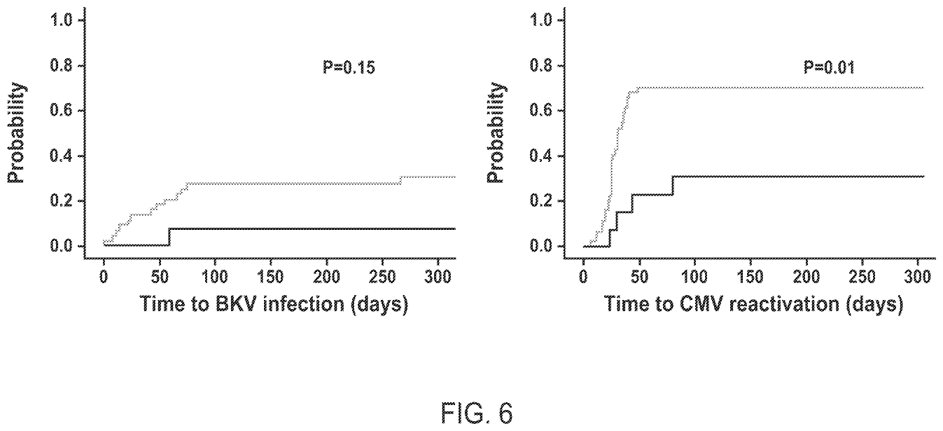

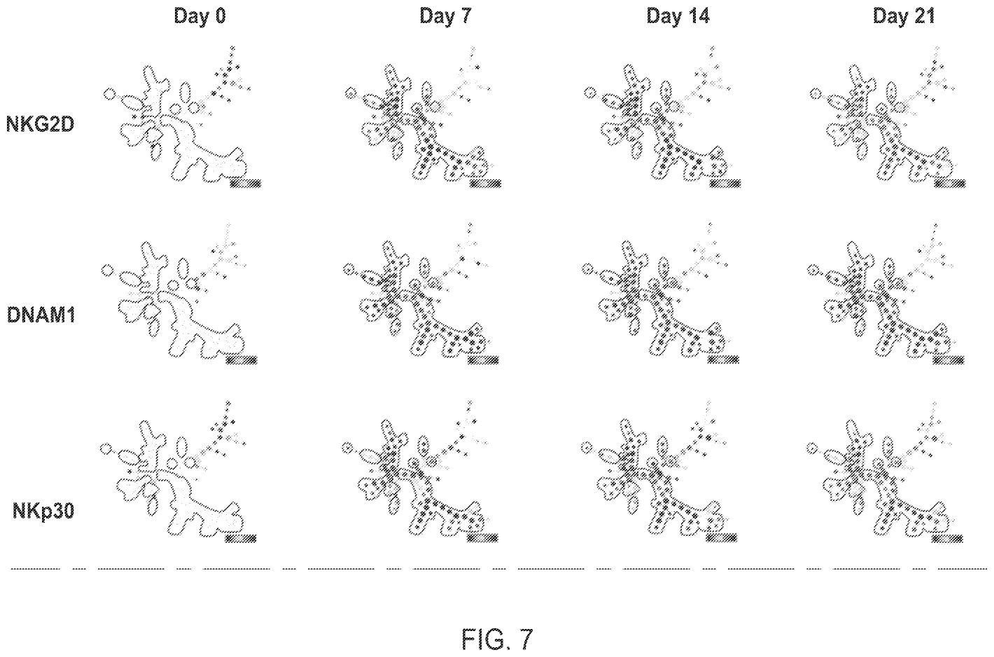

NK cells produced utilizing the mbIL21 feeder cell platform described above have been and continue to be used in clinical trials throughout the world. These trials include 3 completed Phase I clinical trials in multiple myeloma (NCT01729091), AML/MDS (NCT01904136), and myeloid malignancies undergoing matched allogeneic transplant (NCT01823198). In addition to these adult trials, expanded NK cells are being utilized in multiple pediatric trials treating solid tumors such as neuroblastoma (NCT02573896, NCT03242603 and NCT03209869) and pediatric brain tumors (NCT02271711). However, there have not been any trials specifically using these expanded NK cells to combat viral infection to date. In trial NCT01904136 listed above, expanded NK cells were added to an allogeneic hematopoietic stem cell transplant for high-risk myeloid malignancies. There was significantly less viral activation in the post-transplant setting in patients who had received NK cells compared to patients treated on a similar protocol without NK cells (FIG. 6). Importantly, during this and multiple other ongoing and completed trials with autologous and allogeneic NK cell infusion, over 300 infusions of expanded, activated NK cells have been administered to over 100 patients at doses up to 3×10⁸ cells/kg with no dose limiting toxicities.

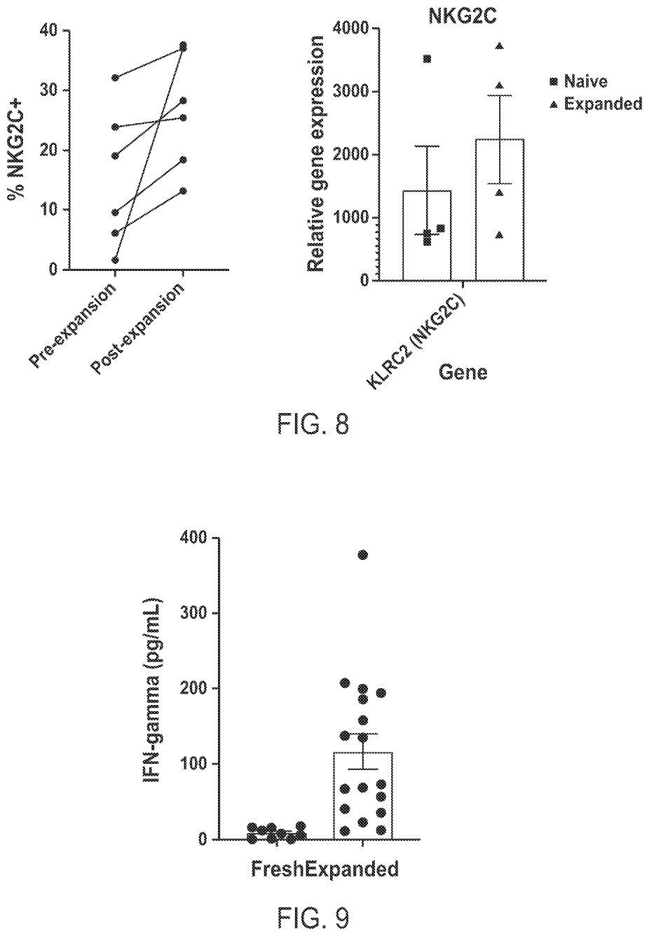

Immunocompetent CMV-seropositive individuals with a previous history of CMV infection have an increased number of circulating NK cells expressing the activating receptor NKG2C and fewer NK cells expressing the inhibitory receptor NKG2A, in response to their persisting CMV infection. Expansion of NKG2C+ NK cells is also observed after Hantavirus and Chikungunya infections, as well as in HIV positive individuals co-infected with CMV. In CMV-seronegative people, the frequency of NK cells expressing NKG2C is low or absent, whereas those who are CMV-seropositive typically have NKG2C expression at 5% or greater, which is further increased with expansion (FIG. 8). No data are available on correlation between CMV status and COVID-19 disease severity yet. CMV seroprevalence is over 95% in the overall Chinese population, 56.7% in Germany (with a higher seroprevalence in women than in men) and 45% in the Netherlands.

Importantly, this invention was advanced to a universal-donor approach and received FDA approval of IND application for manufacturing and clinical testing. The first clinical grade products for infusion were received, which are now available for human use. Donors are identified by Be The Match Biotherapies, and thousands of donors with these universal-donor characteristics can be readily identified. Apheresis of a single donor can generate 10¹² NK cells in 2 weeks.

### Example 5

### Preclinical Models

Parallel methodologies have been developed to generate canine NK cells for comparative medicine models. The first canine expanded NK cells were infused in into a pet dog with osteosarcoma. Importantly, dogs can acquire COVID-19 and therefor represent an important large animal model of the disease and this therapy. Cryopreserved canine NK cells are available for testing.

### Example 6

### Uses of Treating Human Infection

The current mortality rate from 2019-nCoV is 3.2%. This means that nearly 97% of patients are able to mount a successful immune response to 2019-nCoV. Data from SARS-CoV have demonstrated a defect in innate immunity for patients with severe disease that is both quantitative and phenotypic. Adoptive transfer of expanded NK cells augments native immunity in high-risk patients, improving their survival. The invention can be used to treat patients of advanced age, with immunodeficiencies (innate or acquired), with cancer, with cardiorespiratory comorbidities that can make survival from severe 2019-nCoV unlikely, and patients with advanced 2019-nCoV disease (severe respiratory distress with need for at least non-invasive positive pressure respiratory support) with 10⁸ NK cells/kg as a single infusion.

The disclosed invention can be directed towards high-risk individuals diagnosed early in the course of disease with poor immune function, particularly those with low lymphocyte counts (primary immunodeficiency, recent chemotherapy, or solid organ or hematopoietic stem cell transplant). One can consider initial or repeated dosing based on absolute lymphocyte counts (ALC), as low ALC correlates with poor outcomes.

### Example 7

### Preclinical Assessments

The cytolytic effect of ex vivo expanded NK cells against 2019-nCoV infected airway epithelial cells is studied in a pre-clinical setting. Cytolytic activity against this target is measured by Calcein-release cytotoxicity assay. In a BSL3 certified laboratory, the ex vivo expanded NK cells isolated from healthy donors are co-cultured with either health or 2019-nCoV infected airway epithelial cells in in the presence or absence of human serum collected from healthy or 2019-nCoV infected individuals.

### REFERENCES

Barrow A D, Martin C J, Colonna M. The Natural Cytotoxicity Receptors in Health and Disease. Front Immunol. 2019;
Boerman G H et al. Role of NKG2D, DNAM-1 and natural cytotoxicity receptors in cytotoxicity toward rhabdomyosarcoma cell lines mediated by resting and IL-15 activated human natural killer cells. Cancer Immunol Immunother. 2015:64(5):573-83.
Cinzia F, Soriani A, Singoli A, Santoni A, Cippitelli M. NKG2DD and DNAM-1 ligands: molecular targets for NK cell-mediated immunotherapeutic intervention in multiple myeloma. Biomed Res Int. 2015; doi: 10.1155/2015/178698.
Ciurea S O, Schafer J R, Bassett R, Denman C J, Cao K, Willis D, Rondon G, Chen J, Soebbing D, Kaur I, Gulbis A, Ahmed S, Rezvani K, Shpall E J, Lee D A, Champlin R E. Phase 1 clinical trial using mbIL21 ex vivo-expanded donor-derived NK cells after haploidentical transplantation. Blood. 2017; 130(16): 1857-68.
Denman C J, Senyokov V V, Somanchi S S, Phatarpekar P V, Kopp L M, Johnson J L, Singh H, Burton L, Matti S N, Huls M H, Champlin R E, Cooper L J, Lee D A. Membrane-bound IL-21 promotes sustained ex vivo proliferation of human natural killer cells. PLoS One. 2012; 7(1):e30264.
Duev-Cohen A, Bar-On Y, Glasner A, Berhani O, Ophir Y, Levi-Schaffer F, Mandelboim M, Mandelboim O. The human 2B4 and NTB-A receptors bind the influenza viral hemagglutinin and co-stimulate NK cell cytotoxicity. Oncotarget. 2016;7(11):13093-105.
Fernandez L et al. Activated and expanded natural killer cells target osteosarcoma tumor initiating cells in an NKG2D-NKG2DL dependent manner. Cancer Lett. 2015; 368(1):54-63.
Ho J W, Hershkovitz O, Peiris M, Zilka A, Bar-Ilan A, Nal B, et al. H5-type influenza virus hemagglutinin is functionally recognized by the natural killer-activating receptor NKp44. J Virol. 2008; 82:2028-32.
Huang C, et al. Clinical features of patients infected with 2019 novel coronavirus in Wuhan, China. Lancet. 2020; 395:497-506.
Jarahian M, Fielder M, Cohnen A, Djandji D, Hammerling G J, Gati C, et al. Modulation of NKp30- and NKp46-mediated natural killer cell responses by pox viral hemagglutinin. PLoS Pathog. 2011; 7:e1002195.
Jarahian M, Watzl C, Fournier P, Arnold A, Djandji D, Zahedi S, et al. Activation of natural killer cells by Newcastle disease hemagglutinin-neuraminidase. J Virol. 2009; 83:8108-21.
Jernigan D B. Update: Public health response to the coronavirus disease—United States, Feb. 24, 2020. Accessed: Feb. 26, 2020.
Keissling R. Klein E, Pross H, Wigzell H. “Natural” killer cells in the mouse. I. Cytotoxici cells with specificity for mouse Moloney leukemia cells. Specificity and distribution according to genotype. Eur J Immunol. 1975; 5(2):112-7.
Kim j, Yang Y L, Jang S H, Jang Y S. Human β-defensin 2 plays a regulatory role in innate antiviral immunity and is capable of potentiating the induction of antigen-specific immunity. Virol J. 2018; 15(1):124.
Lee D A. Cellular therapy: Adoptive immunotherapy with expanded natural killer cells. Immunol Rev. 2019; 00:1-15.
Ljunggren H G and Karre K. In search of the ‘missing self’: MCH molecules and NK cell recognition. Immunol Today. 1990; 11(7):237-44.
Mahapatra S, et al. High-resolution phenotyping identifies NK cell subsets that distinguish healthy children from adults. PLoS One. 2017; 12(8):e0181134.
Mandelboim O, Lieberman N, Lev M, Paul L, Anion T I, Bushkin Y, et al. Recognition of haemagglutinins on virus-infected cells by NKp46 activates lysis by human NK cells. Nature. 2001; 409:1055-60.
National Research Project for SARS, Beijing Group. The involvement of natural killer cells in the pathogenesis of Severe Acute Respiratory Syndrome. Am J Clin Pathol. 2004; 121:507-511.
Sainz B, Mossel, E C, Peters C J, Garry R F. Interferon-beta and interferon-gamma synergistically inhibit the replication of severe acute respiratory syndrome-associated coronavirus (SARS-CoV). Virology. 2004; 329(1):11-7.
Shah N, Li L, McCarty J, Kaur I, Yvon E, Shaim H, Muftuoglu M, Liu E, Orlowski R Z, Cooper L, Lee D, Parmar S, Cao K, Sobieski C, Saliba R, Hosin C, Ahmed S, Neito Y, Bashir Q, Patel K, Bollard C, Qazilbash M, Champlin R, Rezvani K, Shpall E J. Phase 1 study of cord blood-derived natural killer cells combined with autologous stem cell transplantation in multiple myeloma. Br J Haematol. 2017; 177(3):457-66.
Single-cell RNA expression profiling of ACE2, the putative receptor of Wuhan 2019-nCov. BioRxiv. 2020; doi:.
Somanchi S S, Senyukov V V, Denman C J, Lee D A. Expansion, purification and functional assessment of human peripheral blood NK cells. J Vis Exp 2011; 48:2540.
Tian X, et al. Potent binding of 2019 novel coronavirus spike protein by a SARS coronavirus-specific human monoclonal antibody. Emerging Microbes and Infections; 2020; 9(1):382-5.
Verhoeven D H J et al. NK cells recognize and lyse Ewing sarcoma cells through NKG2D and DNAM-1 receptor dependent pathways. Mol Immunol. 2008; 45(15):3917-25.
Wang X, Lee D A, Wang Y, Wang L, Yao Y, Lin Z, Cheng J, Zhu S. Membrane-bound interleukin-21 and CD137 ligand induce functional human natural killer cells from peripheral blood mononuclear cells through STAT-3 activation. Clin Exp Immunol. 2013; 172(1):104-12.
World Health Organiziation (WHO). Coronavirus. Geneva: WHO; 2020.
Xu X et al. Evolution of the novel coronavirus from the ongoing Wuhan outbreak and modeling of its spike protein for risk of human transmission. Science China Life Sciences. 2020; 63(3):457-60.
Zhang Y Z. Novel 2019 coronavirus genome. Viorological.

What is claimed is:

1. A method of treating a microbial infection in a subject comprising administering to the subject a therapeutically effective amount of expanded natural killer (NK) cells.

2. The method treating a microbial infection of claim 1, further comprising obtaining a nonexpanded, nonactivated NK cell and expanding the nonexpanded, nonactivated NK cell through contacting the nonexpanded, nonactivated NK cell with IL-21, IL-15, and/or 4-BBL.

3. The method treating a microbial infection of claim 2, wherein the IL-21, IL-15, and/or 4-1BBL are soluble.

4. The method treating a microbial infection of claim 2, wherein the IL-21, IL-15, and/or 4-1BBL are expressed on the surface of an engineered plasma membrane vesicle, an engineered exosome, an engineered liposome, or an engineered feeder cell; wherein said engineered plasma membrane vesicle, an engineered exosome, an engineered liposome, or an engineered feeder cell is engineered to express membrane bound IL-21 (mbIL-21), IL-15 (mbIL-15), and/or 4-1BBL (mb4-1BBL).

5. The method treating a microbial infection of any of claims 2-4, wherein the expansion of the nonexpanded, nonactivated NK cell occurs ex vivo.

6. The method treating a microbial infection of any of claims 2-4, wherein the expansion of the nonexpanded, nonactivated NK cell occurs in vivo.

7. The method treating a microbial infection of any one of claims 4-6, wherein the engineered plasma membrane vesicle, exosome, or feeder cell are derived from feeder cells selected from the group consisting of peripheral blood mononuclear cell (PBMC), RPMI8866, NK-92, NK-92MI, NK-YTS, NK, NKL, KIL, KIL C.2, NK 3.3, NK-YS, HFWT, K562, and/or EBV-LCL cells.

8. The method treating a microbial infection of any one of claims 2-7, wherein the nonexpanded, nonactivated NK cell comprises a primary NK cell, CAR-NK cell, memory-like NK cell, or an NK cell line.

9. The method treating a microbial infection of any one of claims 1-8, wherein the expanded NK cells comprise increased expression levels of one or more NK cell receptors selected from group consisting of KIR2DL2, NKp46, NKp44, NKp30, CD226, NKG2D, 2B4, CD11a, OX40, 4-1BB, CD223, and ICOS.

10. The method treating a microbial infection of any one of claims 1-8, wherein the expanded NK cells comprises increased expression levels of one or more anti-microbial effectors selected from group consisting of granzyme B, TNFα, IFNγ, and perforin.

11. The method treating a microbial infection of any one of claims 1-10, wherein the expanded NK cells comprise autologous, haploidentical, or allogeneic NK cells.

12. The method treating a microbial infection of any one of claims 1-11, wherein the microbial infection comprises a viral infection, bacterial infection, fungal infection, or parasitic infection.

13. The method treating a microbial infection of claim 12, wherein the viral infection comprises an infection of coronavirus, herpesvirus, polyomavirus, or influenza.

14. The method treating a microbial infection of claim 13, wherein the coronavirus is 2019-nCoV, severe acute respiratory syndrome-related coronavirus (SARS), or Middle East respiratory syndrome-related coronavirus (MERS).

15. A method of generating expanded natural killer (NK) cells that comprise increased expression levels of KIR2DL2, comprising obtaining a nonexpanded, nonactivated NK cell and expanding the nonexpanded, nonactivated NK cell through contacting the nonexpanded, nonactivated NK cell with IL-21, IL-15, and/or 4-BBL.

16. The method generating expanded natural killer (NK) cells that comprise increased expression levels of KIR2DL2 of claim 15, wherein the IL-21, IL-15, and/or 4-1BBL are soluble.

17. The method generating expanded natural killer (NK) cells that comprise increased expression levels of KIR2DL2 of claim 15, wherein the IL-21, IL-15, and/or 4-1BBL are expressed on the surface of an engineered plasma membrane vesicle, an engineered exosome, an engineered liposome, or an engineered feeder cell; wherein said engineered plasma membrane vesicle, an engineered exosome, an engineered liposome, or an engineered feeder cell is engineered to express membrane bound IL-21 (mbIL-21), IL-15 (mbIL-15), and/or 4-1BBL (mb4-1BBL).

18. The method generating expanded natural killer (NK) cells that comprise increased expression levels of KIR2DL2 of any of claims 15-17, wherein the expansion of the nonexpanded, nonactivated NK cell occurs ex vivo.

19. The method generating expanded natural killer (NK) cells that comprise increased expression levels of KIR2DL2 of any of claims 15-17, wherein the expansion of the nonexpanded, nonactivated NK cell occurs in vivo.

20. The method generating expanded natural killer (NK) cells that comprise increased expression levels of KIR2DL2 of any of claims 17-19, wherein the engineered plasma membrane vesicle, exosome, or feeder cell are derived from feeder cells selected from the group consisting of peripheral blood mononuclear cell (PBMC), RPMI8866, NK-92, NK-92MI, NK-YTS, NK, NKL, KIL, KIL C.2, NK 3.3, NK-YS, HFWT, K562, and/or EBV-LCL cells.

21. The method of any one of claims 15-20, wherein the nonexpanded, nonactivated NK cell comprises a primary NK cell, CAR-NK cell, memory-like NK cell, or an NK cell line.

22. The method of any one of claims 15-21, wherein the expanded NK cells comprise increased expression levels of one or more NK cell receptors selected from group consisting of KIR2DL2, NKp46, NKp44, NKp30, CD226, NKG2D, 2B4, CD11a, OX40, 4-1BB, CD223, and ICOS.

23. The method of any one of claims 15-22, wherein the expanded NK cells comprise increased expression levels of one or more anti-microbial effectors selected from group consisting of granzyme B, TNFα, IFNγ, and perforin.

24. The method of any one of claims 15-23, further comprising administering a therapeutically effective amount of the expanded NK cells to a subject in need thereof for treating a microbial infection.

25. The method of claim 15-24, wherein the expanded NK cells comprise autologous, haploidentical, or allogeneic NK cells.

26. A method of increasing the expression level of KIR2DL2 in a natural killer (NK) cell comprising obtaining a NK cell and expanding the NK cell through contacting the NK cell with IL-21, IL-15, and/or 4-BBL.

27. The method increasing the expression level of KIR2DL2 in a NK cell of claim 26, wherein the IL-21, IL-15, and/or 4-1BBL are soluble.

28. The method increasing the expression level of KIR2DL2 in a NK cell of claim 26, wherein the IL-21, IL-15, and/or 4-1BBL are expressed on the surface of an engineered plasma membrane vesicle, an engineered exosome, an engineered liposome, or an engineered feeder cell; wherein said engineered plasma membrane vesicle, an engineered exosome, an engineered liposome, or an engineered feeder cell is engineered to express membrane bound IL-21 (mbIL-21), IL-15 (mbIL-15), and/or 4-1BBL (mb4-1BBL).

29. The method increasing the expression level of KIR2DL2 in a NK cell of any of claims 26-28, wherein the expansion of the nonexpanded, nonactivated NK cell occurs ex vivo.

30. The method increasing the expression level of KIR2DL2 in a NK cell of any of claims 26-28, wherein the expansion of the nonexpanded, nonactivated NK cell occurs in vivo.

31. The method increasing the expression level of KIR2DL2 in a NK cell of any of claims 26-30, wherein the engineered plasma membrane vesicle, exosome, or feeder cell are derived from feeder cells selected from the group consisting of peripheral blood mononuclear cell (PBMC), RPMI8866, NK-92, NK-92MI, NK-YTS, NK, NKL, KIL, KIL C.2, NK 3.3, NK-YS, HFWT, K562, and/or EBV-LCL cells.

32. The method increasing the expression level of KIR2DL2 in a NK cell of any of claims 26-31, wherein the nonexpanded, nonactivated NK cell comprises a naive NK cell, a primary NK cell, CAR-NK cell, memory-like NK cell, or an NK cell line.

33. A preclinical method of examining an NK cell adoptive immunotherapy for treating 2019-nCoV infection, comprising
a) administering expanded NK cells to a canine that is infected with 2019-nCoV; and
b) determining that the expanded NK cells are effective if the viral titers of 2019-nCoV in the canine decrease.

34. The preclinical method of claim 33, further comprising obtaining a nonexpanded, nonactivated NK cell and expanding the nonexpanded, nonactivated NK cell through contacting the nonexpanded, nonactivated NK cell with a plasma membrane vesicle, an exosome, or a feeder cell that is engineered to express membrane bound IL-21.

35. The preclinical method of claim 34, wherein the expansion of the nonexpanded, nonactivated NK cell occurs ex vivo.

36. The preclinical method of claim 34, wherein the expansion of the nonexpanded, nonactivated NK cell occurs in vivo.

37. The method of any one of claims 33-36, wherein the nonexpanded, nonactivated NK cell comprises a primary NK cell or an NK cell line.

38. The method of any one of claims 33-37, wherein the expanded NK cells comprise increased expression levels of one or more NK cell receptors selected from group consisting of KIR2DL2, NKp46, NKp44, NKp30, CD226, NKG2D, 2B4, CD11a, OX40, 4-1BB, CD223, and ICOS.

39. The method of any one of claims 33-37, wherein the expanded NK cells comprises increased expression levels of one or more anti-microbial effectors selected from group consisting of granzyme B, TNFα, IFNγ, and perforin.

40. The method of any one of claims 33-39, wherein the expanded NK cells comprise autologous, haploidentical, or allogeneic NK cells.

41. A preclinical method of examining an NK cell adoptive immunotherapy for treating 2019-nCoV infection comprising a canine the expanded canine NK cells generated by the methods of any of claims 33-40.
